(19) United States
(12) Patent Application Publication
(10) Pub. No.: US 2008/0254140 A1
(43) Pub. Date: Oct. 16, 2008

(54) Combination Methods and Therapies for Treating Opthalmic Conditions with 13-Cis-Retinyl Derivatives

(76) Inventors: Kenneth Widder, Rancho Santa Fe, CA (US); Jay Lichter, San Diego, CA (US)

Correspondence Address: WILSON SONSINI GOODRICH & ROSATI, 650 PAGE MILL ROAD, PALO ALTO, CA, 94304-1050, US

(21) Appl. No.: 11/659,915

(22) PCT Filed: Aug. 18, 2005
(86) PCT No.: PCT/US2005/029455

§ 371 Date: Feb. 21, 2008

Related U.S. Application Data
(60) Provisional application No. 60/602,675, filed on Aug. 18, 2004.

(51) Int. Cl. A61K 33/34; A61K 31/164; A61K 31/215; A61K 31/095; A61K 31/12; A61K 33/30; A61K 31/66; A61P 27/02; A61K 33/04; A61K 31/122; A61K 31/445; A61K 31/355; A61K 36/45

(52) U.S. Cl. 424/630; 514/613; 514/529; 514/712; 514/690; 514/688; 514/315; 514/458; 424/732; 424/641; 424/702; 514/121

(57) ABSTRACT

Described herein are combination methods, compositions and therapies for treating ophthalmic conditions or diseases arising from, associated with or leading to the overproduction of waste products in the visual cycle. Agents included within these combinations are 13-cis-retinyl derivatives; other agents included within these combinations are selected from vitamins, antioxidants, minerals, inducers of nitric oxide production, anti-inflammatory agents, and negatively-charged phospholipids. Such combination methods may be used as single or multiple administration therapies, or in combination with other agents or therapies.

### BACKGROUND OF THE INVENTION

The visual cycle or retinoid cycle is a series of light-driven and enzyme catalyzed reactions in which the active visual chromophore rhodopsin is converted to an all-trans-isomer that is subsequently regenerated. Part of the cycle occurs within the outer segment of the rods and part of the cycle occurs in the retinal pigment epithelium (RPE). Components of this cycle include various dehydrogenases and isomerases, as well as proteins for transporting intermediates between the photoreceptors and the RPE.

Other proteins associated with the visual cycle are responsible for transporting, removing and/or disposing compounds and toxic products that accumulate from excess production of visual cycle retinoids, such as all-trans-retinal. An example of such a compound is the diretinal species N-retinylidene-N-retinylethanolamine (A2E), which arises from the condensation of all-trans-retinal with phosphatidylethanolamine. Although certain levels of this orange-emitting fluorophore are tolerated by the photoreceptors and the RPE, excessive quantities can lead to adverse effects, including the production of lipofuscin and potentially drusen under the macula. See, e.g., Finnemann, S. C., Proc. Natl. Acad. Sci., 99:3842-47 (2002). Drusen are extracellular deposits that accumulate below the RPE and are risk factors for developing age-related macular degeneration. See, e.g., Crabb, J. W., et al., Proc. Natl. Acad. Sci., 99:14682-87 (2002). Thus, removal and disposal of compounds and toxic products that arise from side reactions in the visual cycle is important because several lines of evidence indicate that the over-accumulation of such compounds and toxic products may be partially responsible for the symptoms associated with the macular degenerations and dystrophies.

There are two general categories of age-related macular degeneration: the wet and dry forms. Dry macular degeneration, which accounts for about 90 percent of all cases, is also known as atrophic, nonexudative, or drusenoid macular degeneration. With dry macular degeneration, drusen typically accumulate in the RPE tissue beneath the macula. Vision loss can then occur when drusen interfere with the function of photoreceptors in the macula. This form of macular degeneration results in the gradual loss of vision over many years.

Wet macular degeneration, which accounts for about 10 percent of cases, is also known as choroidal neovascularization, subretinal neovascularization, exudative, or disciform degeneration. In wet macular degeneration, abnormal blood vessel growth can form beneath the macula; these vessels can leak blood and fluid into the macula and damage photoreceptor cells. Studies have shown that advanced stages of the dry form of macular degeneration can lead to the wet form of macular degeneration. The wet form of macular degeneration can progress rapidly and cause severe damage to central vision.

Stargardt Disease, also known as Stargardt Macular Dystrophy or Fundus Flavimaculatus, is the most frequently encountered juvenile onset form of macular dystrophy. Research indicates that this condition is transmitted as an autosomal recessive trait in the ABCR gene. This gene is a member of the ABC Super Family of genes that encode for transmembrane proteins involved in the energy dependent transport of a wide spectrum of substances across membranes. Stargardt-like macular dystrophy is a dominant inherited trait involving loss of central vision, but it begins later than Stargardt macular dystrophy, and the accumulation of lipofuscsin extends beyond the central region of the macula.

Symptoms of Stargardt Macular Dystrophy include a decrease in central vision and difficulty with dark adaptation, problems that generally worsen with age so that many persons afflicted with Stargardt Macular Dystrophy experience visual loss of 20/100 to 20/400 by 30 to 40 years of age. Persons with Stargardt Macular Dystrophy are generally encouraged to avoid bright light because of the potential over-production of all-trans-retinal.

Methods for diagnosing Stargardt Macular Dystrophy include the observation of an atrophic or “beaten-bronze” appearance of deterioration in the macula, and the presence of numerous yellowish-white spots that occur within the retina surrounding the atrophic-appearing central macular lesion. Other diagnostic tests include the use of an electroretinogram, electro-oculogram, and dark adaptation testing. In addition, a fluorescein angiogram can be used to confirm the diagnosis. In this latter test, observation of a “dark” or “silent” choroid appears associated with the accumulation of lipofuscin in the retinal pigment epithelium of the patient, one of the early symptoms of macular degeneration.

Currently, treatment options for the macular degenerations and macular dystrophies are limited. Some patients with dry form AMD have responded to high doses of vitamins and minerals. In addition, a few studies have indicated that laser photocoagulation of drusen may prevent or delay the development of drusen that can lead to the more severe symptoms of dry form AMD. Finally, certain studies have shown that extracorporeal rheopheresis may provide benefit to patients with dry form AMD.

However, successes have been limited and there continues to be a strong desire for new methods and treatments to manage and limit vision loss associated with the macular degenerations and dystrophies.

### SUMMARY OF THE INVENTION

Presented herein are combination methods and formulations for (a) treating ophthalmic conditions, and (b) controlling symptoms that presage (e.g., risk factors) or are associated with such ophthalmic conditions. In one aspect, such combination methods and formulations comprise the use of retinyl derivatives with an additional agent. In other aspects the ophthalmic conditions are macular degenerations (including, but not limited to the dry form and the we form) and macular dystrophies (including but not limited to Stargardt Disease and Stargardt-like macular dystrophy). In other aspects, the methods and formulations are used to protect eyes of a mammal from light; in other aspects the methods and formulations are used to limit the formation of all-trans-retinal, N-retinylidene-N-retinylethanolamine, lipofuscin and/or drusen in the eye of a mammal. In yet other aspects, the combination methods and formulations are used in further combinations with other treatment modalities.

In one aspect is a method for reducing the formation of all-trans-retinal in an eye of a mammal comprising administering to the mammal at least once an effective amount of a first agent, wherein the first agent has the structure of Formula (I):

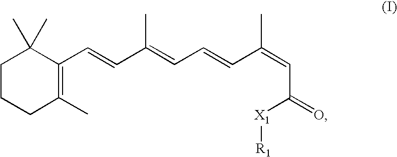

wherein X1 is selected from the group consisting of NR2, O, S, CHR2; R1 is (CHR2)x-L1-R3, wherein x is 0, 1, 2, or 3; L1 is a single bond or —C(O)—; R2 is a moiety selected from the group consisting of H, (C1-C4)alkyl, F, (C1-C4)fluoroalkyl, (C1-C4)alkoxy, —C(O)OH, —C(O)—NH2, —(C1-C4)alkylamine, —C(O)—(C1-C4)alkyl, —C(O)—(C1-C4)fluoralkyl, —C(O)—(C1-C4)alkylamine, and —C(O)—(C1-C4)alkoxy; and R3 is H or a moiety, optionally substituted with 1-3 independently selected substituents, selected from the group consisting of (C2-C7)alkenyl, (C2-C7)alkynyl, aryl, (C3-C7)cycloalkyl, (C5-C7)cycloalkenyl, and a heterocycle; or an active metabolite, or a pharmaceutically acceptable prodrug or solvate thereof; and an effective amount of a second agent comprising an agent selected from the group consisting of an antioxidant, a mineral, an inducer of nitric oxide production, an anti-inflammatory agent, and a negatively charged phospholipid.

In another aspect is a method for reducing the formation of all-trans-retinal in an eye of a mammal comprising administering to the mammal at least once (a) an effective amount of a compound selected from the group consisting of 13-cis retinoic acid, isosteres of 13-cis retinoic acid, prodrugs of 13-cis retinoic acid, tautomers of 13-cis retinoic acid, protected forms of 13-cis retinoic acids thereof and (b) an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their ester and amide derivatives (including by way of example only, all-trans retinoic acid, also known as tretinoin, and fenretinide, respectively).

In another aspect is a method for reducing the formation of N-retinylidene-N-retinylethanolamine in an eye of a mammal comprising administering to the mammal at least once an effective amount of a first agent, wherein the first agent has the structure of Formula (I):

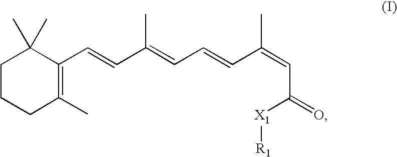

wherein X1 is selected from the group consisting of NR2, O, S, CHR2; R1 is (CHR2)x-L1-R3, wherein x is 0, 1, 2, or 3; L1 is a single bond or —C(O)—; R2 is a moiety selected from the group consisting of H, (C1-C4)alkyl, F, (C1-C4)fluoroalkyl, (C1-C4)alkoxy, —C(O)OH, —C(O)—NH2, —(C1-C4)alkylamine, —C(O)—(C1-C4)alkyl, —C(O)—(C1-C4)fluoralkyl, —C(O)—(C1-C4)alkylamine, and —C(O)—(C1-C4)alkoxy; and R3 is H or a moiety, optionally substituted with 1-3 independently selected substituents, selected from the group consisting of (C2-C7)alkenyl, (C2-C7)alkynyl, aryl, (C3-C7)cycloalkyl, (C5-C7)cycloalkenyl, and a heterocycle; or an active metabolite, or a pharmaceutically acceptable prodrug or solvate thereof; and an effective amount of a second agent comprising an agent selected from the group consisting of an antioxidant, a mineral, an inducer of nitric oxide production, an anti-inflammatory agent, and a negatively charged phospholipid.

In another aspect is a method for reducing the formation of N-retinylidene-N-retinylethanolamine in an eye of a mammal comprising administering to the mammal at least once an (a) an effective amount of a compound selected from the group consisting of 13-cis retinoic acid, isosteres of 13-cis retinoic acid, prodrugs of 13-cis retinoic acid, tautomers of 13-cis retinoic acid, protected forms of 13-cis retinoic acids thereof and (b) an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their ester and amide derivatives (including by way of example only, all-trans retinoic acid, also known as tretinoin, and fenretinide, respectively).

In another aspect is a method for reducing the formation of lipofuscin in an eye of a mammal comprising administering to the mammal at least once an effective amount of a first agent, wherein the first agent has the structure of Formula (I):

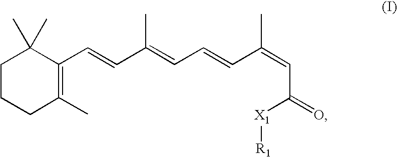

wherein X1 is selected from the group consisting of NR2, O, S, CHR2; R1 is (CHR2)x-L1-R3, wherein x is 0, 1, 2, or 3; L1 is a single bond or —C(O)—; R2 is a moiety selected from the group consisting of H, (C1-C4)alkyl, F, (C1-C4)fluoroalkyl, (C1-C4)alkoxy, —C(O)OH, —C(O)—NH2, —(C1-C4)alkylamine, —C(O)—(C1-C4)alkyl, —C(O)—(C1-C4)fluoralkyl, —C(O)—(C1-C4)alkylamine, and —C(O)—(C1-C4)alkoxy; and R3 is H or a moiety, optionally substituted with 1-3 independently selected substituents, selected from the group consisting of (C2-C7)alkenyl, (C2-C7)alkynyl, aryl, (C3-C7)cycloalkyl, (C5-C7)cycloalkenyl, and a heterocycle; or an active metabolite, or a pharmaceutically acceptable prodrug or solvate thereof; and an effective amount of a second agent comprising an agent selected from the group consisting of an antioxidant, a mineral, an inducer of nitric oxide production, an anti-inflammatory agent, and a negatively charged phospholipid.

In another aspect is a method for reducing the formation of lipofuscin in an eye of a mammal comprising administering to the mammal at least once an (a) an effective amount of a compound selected from the group consisting of 13-cis retinoic acid, isosteres of 13-cis retinoic acid, prodrugs of 13-cis retinoic acid, tautomers of 13-cis retinoic acid, protected forms of 13-cis retinoic acids thereof and (b) an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their ester and amide derivatives (including by way of example only, all-trans retinoic acid, also known as tretinoin, and fenretinide, respectively).

In another aspect is a method for reducing the formation of drusen in an eye of a mammal comprising administering to the mammal at least once an effective amount of a first agent, wherein the first agent has the structure of Formula (I):

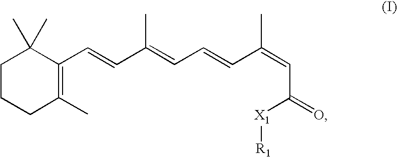

wherein X1 is selected from the group consisting of NR2, O, S, CHR2; R1 is (CHR2)x-L1-R3, wherein x is 0, 1, 2, or 3; L1 is a single bond or —C(O)—; R2 is a moiety selected from the group consisting of H, (C1-C4)alkyl, F, (C1-C4)fluoroalkyl, (C1-C4)alkoxy, —C(O)OH, —C(O)—NH2, —(C1-C4)alkylamine, —C(O)—(C1-C4)alkyl, —C(O)—(C1-C4)fluoralkyl, —C(O)—(C1-C4)alkylamine, and —C(O)—(C1-C4)alkoxy; and R3 is H or a moiety, optionally substituted with 1-3 independently selected substituents, selected from the group consisting of (C2-C7)alkenyl, (C2-C7)alkynyl, aryl, (C3-C7)cycloalkyl, (C5-C7)cycloalkenyl, and a heterocycle; or an active metabolite, or a pharmaceutically acceptable prodrug or solvate thereof, and an effective amount of a second agent comprising an agent selected from the group consisting of an antioxidant, a mineral, an inducer of nitric oxide production, an anti-inflammatory agent, and a negatively charged phospholipid.

In another aspect is a method for reducing the formation of drusen in an eye of a mammal comprising administering to the mammal at least once an (a) an effective amount of a compound selected from the group consisting of 13-cis retinoic acid, isosteres of 13-cis retinoic acid, prodrugs of 13-cis retinoic acid, tautomers of 13-cis retinoic acid, protected forms of 13-cis retinoic acids thereof and (b) an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their ester and amide derivatives (including by way of example only, all-trans retinoic acid, also known as tretinoin, and fenretinide, respectively).

In another aspect is a method for protecting the photoreceptors in any eye of a mammal comprising administering to the mammal at least once an effective amount of a first agent, wherein the first agent has the structure of Formula (I):

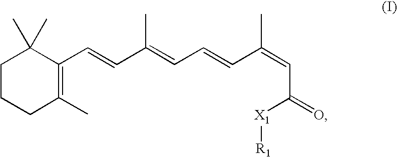

wherein X1 is selected from the group consisting of NR2, O, S, CHR2; R1 is (CHR2)x-L1-R3, wherein x is 0, 1, 2, or 3; L1 is a single bond or —C(O)—; R2 is a moiety selected from the group consisting of H, (C1-C4)alkyl, F, (C1-C4)fluoroalkyl, (C1-C4)alkoxy, —C(O)OH, —C(O)—NH2, —(C1-C4)alkylamine, —C(O)—(C1-C4)alkyl, —C(O)—(C1-C4)fluoralkyl, —C(O)—(C1-C4)alkylamine, and —C(O)—(C1-C4)alkoxy; and R3 is H or a moiety, optionally substituted with 1-3 independently selected substituents, selected from the group consisting of (C2-C7)alkenyl, (C2-C7)alkynyl, aryl, (C3-C7)cycloalkyl, (C5-C7)cycloalkenyl, and a heterocycle; or an active metabolite, or a pharmaceutically acceptable prodrug or solvate thereof; and an effective amount of a second agent comprising an agent selected from the group consisting of an antioxidant, a mineral, an inducer of nitric oxide production, an anti-inflammatory agent, and a negatively charged phospholipid.

In another aspect is a method for protecting the photoreceptors in any eye of a mammal comprising administering to the mammal at least once an (a) an effective amount of a compound selected from the group consisting of 13-cis retinoic acid, isosteres of 13-cis retinoic acid, prodrugs of 13-cis retinoic acid, tautomers of 13-cis retinoic acid, protected forms of 13-cis retinoic acids thereof and (b) an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their ester and amide derivatives (including by way of example only, all-trans retinoic acid, also known as tretinoin, and fenretinide, respectively).

In yet another aspect is a method for preventing macular degeneration in an eye of a mammal comprising administering to the mammal at least once an effective amount of a first agent, wherein the first agent has the structure of Formula (I):

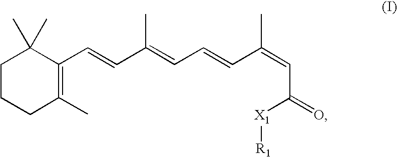

wherein X1 is selected from the group consisting of NR2, O, S, CHR2; R1 is (CHR2)x-L1-R3, wherein x is 0, 1, 2, or 3; L1 is a single bond or —C(O)—; R2 is a moiety selected from the group consisting of H, (C1-C4)alkyl, F, (C1-C4)fluoroalkyl, (C1-C4)alkoxy, —C(O)OH, —C(O)—NH2, —(C1-C4)alkylamine, —C(O)—(C1-C4)alkyl, —C(O)—(C1-C4)fluoralkyl, —C(O)—(C1-C4)alkylamine, and —C(O)—(C1-C4)alkoxy; and R3 is H or a moiety, optionally substituted with 1-3 independently selected substituents, selected from the group consisting of (C2-C7)alkenyl, (C2-C7)alkynyl, aryl, (C3-C7)cycloalkyl, (C5-C7)cycloalkenyl, and a heterocycle; or an active metabolite, or a pharmaceutically acceptable prodrug or solvate thereof; and an effective amount of a second agent comprising an agent selected from the group consisting of an antioxidant, a mineral, an inducer of nitric oxide production, an anti-inflammatory agent, and a negatively charged phospholipid.

In yet another aspect is a method for preventing macular degeneration in an eye of a mammal comprising administering to the mammal at least once an (a) an effective amount of a compound selected from the group consisting of 13-cis retinoic acid, isosteres of 13-cis retinoic acid, prodrugs of 13-cis retinoic acid, tautomers of 13-cis retinoic acid, protected forms of 13-cis retinoic acids thereof and (b) an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their ester and amide derivatives (including by way of example only, all-trans retinoic acid, also known as tretinoin, and fenretinide, respectively).

Further embodiments of any of the aforementioned aspects comprise administration at least once of at least one additional agent comprising an agent selected from the group consisting of an inducer of nitric oxide production, an anti-inflammatory agent, a physiologically acceptable antioxidant, a physiologically acceptable mineral, a negatively charged phospholipid, and 13-cis-retinoic acid. Still further embodiments of any of the aforementioned aspects comprise administering an additional treatment selected from the group consisting of extracorporeal rheopheresis and laser photocoagulation to remove drusen.

In another aspect are pharmaceutical compositions for (a) reducing the formation of N-retinylidene-N-retinylethanolamine in an eye of a mammal, (b) reducing the formation of lipofuscin in an eye of a mammal, (c) reducing the formation of drusen in an eye of a mammal, (d) preventing macular degeneration in an eye of a mammal, and/or (e) reducing the formation of all-trans-retinal in an eye of a mammal, comprising an effective amount of at least one compound having the structure of Formula (I) in combination with an effective amount of a second agent comprising an agent selected from the group consisting of an antioxidant, a mineral, an inducer of nitric oxide production, an anti-inflammatory agent, and a negatively charged phospholipid.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All references cited herein, including patents, patent applications, and publications, are hereby incorporated by reference in their entirety.

### DETAILED DESCRIPTION OF THE INVENTION

Retinoids are compounds that have a varied effect on biological systems such as cellular growth and differentiation, immunomodulation, tumor promotion, and inhibition of cell growth. See, e.g., Grunwald, et al., J. Nucl. Med., 39:1903-6 (1998); Cheng, et al., J. Formos. Med. Assoc., 96:525-34 (1997); Huang, et al., Proc. Natl. Acad. Sci., 94:5826-30 (1997); Yokota et al., Atherosclerosis 159:491-6 (2001). Retinoids are any variety of natural or synthetic derivatives of vitamin A that function by binding receptors that directly and/or indirectly regulate transcription of genes. See, e.g., Goldfarb, et al., Curr. Opin. Dermatol., 4:236-40 (1997). Isotretinoin or 13-cis retinoic acid has been used for the treatment of many dermatologic conditions. See, e.g., Peck, et al., New Engl. J. Med., 300:329-333 (1979). Recently, studies have indicated that isotretinoin treatment slows the formation of 11-cis-retinal which leads to production of all-trans-retinal that may ultimately bring about the loss of photoreceptors. See, e.g., Radu, et al., Proc. Natl. Acad. Sci., 100:4742-47 (2003).

Identity of Second Agents. Second agents can be selected from a number of sources, including, but not limited to an antioxidant, a mineral, an inducer of nitric oxide production, an anti-inflammatory agent, a negatively charged phospholipid, and suitable isomers of 13-cis-retinoic acid and their ester and amide derivatives (including by way of example only, all-trans retinoic acid, also known as tretinoin, and fenretinide, respectively). Additional second agents are also identified throughout the text. It is to be understood that certain second agents may fall within multiple classes of agents. Thus, by way of example only, zinc is both a mineral and an anti-oxidant, and vitamin C is both a vitamin and an anti-oxidant. Thus, the placement of an agent in one category should not be seen as excluding it from another category.

Other studies have been directed to the use of dietary supplements in the treatment of age-related macular degeneration. Such research has provided evidence that high-potency antioxidant vitamin and mineral supplements can slow the progression of moderate to advanced forms of age-related macular degeneration and its associated vision loss. See, e.g., AREDS Report No. 8 Arch. Ophthalmol., 119:1417-36 (2001); Chang, et al. Can. J. Opthalmol., 38:27-32 (2003). In addition, daily intake of particular dietary supplements has been considered important in the healthy maintenance of the eye retina and lens by protecting them from oxidative damage due to free radicals that can be generated by normal metabolic functions as well as exposure to radiation in sunlight or toxic pollutants in the environment. See, e.g., Brown, et al., Eye, 12:127-133 (1998).

In particular, vitamins A, C and E seem to have an effect in the healthy maintenance of the eye. Vitamin A has a role in the formation of the retinal photoreceptor pigments and lack of it can lead to a decrease in night vision. See, e.g., Brown, et al. A high concentration of vitamin C can be found in the aqueous humour which suggests its important role in maintenance of the eye lens. See, e.g., Taylor, et al., Curr. Eye Res., 10:751-9 (1991). Vitamin C also has a role in reducing the development of cataracts and protecting the retina from light damage. See, e.g., Tso, et al., Curr. Eye Res., 3:166-74 (1984); Robertson, et al., Ann. NY Acad. Sci., 570:372-82 (1989). Vitamin E has been implicated in reducing the risk of cortical, nuclear and mixed cataract types. See, e.g., Leske, et al., Arch. Opthalmol., 109:244-51 (1991).

We consider that the compounds of Formula (I) in combination with certain vitamins as a second agent can be used to provide benefit to patients suffering from or susceptible to various macular degenerations and dystrophies, including but not limited to dry-form age-related macular degeneration and Stargardt Disease. That is, we consider compounds of Formula (I) in combination with vitamins to be capable of providing at least some of the following benefits to such human patients: reduction in the amount of all-trans-retinal, reduction in the formation of A2E, reduction in the formation of lipofuscin, reduction in the formation of drusen, and reduction in light sensitivity. In addition, because dry-form age-related macular degeneration is often a precursor to wet-form age-related macular degeneration, the use of compounds of Formula (I) in combination with vitamins can also be used as a preventative therapy for this latter ophthalmic condition.

A combination of at least one compound of Formula (I) and vitamins might not be expected to provide additional benefit beyond the separate use of these therapeutics. In addition, isotretinoin is a derivative of vitamin A, where an overdose of vitamin A has been shown to result in many harmful effects which include acute and chronic toxicity. Acute toxicity can lead to symptoms which include: intracranial hypertension, nausea, vomiting, vertigo, visual disorientation and peeling of the skin. See, e.g., Gangemi et al., Acta Neurol. 7:27-31 (1985); Bendich and Langseth, Am. J. Clin. Nutr. 49:358-371 (1989); Hathcock et al., Am. J. Clin. Nutr. 52:183-202 (1990). Symptoms of chronic vitamin A toxicity have been studied in animals which include: hair loss, localized erythema, thickened epithelium, fatty infiltration of the liver and heart, kidney and testicular defects, anemia, hypercholesterolemia, sometimes hypertriglyceridemia, and skeletal alterations. See, e.g., Singh and Singh, Am. J. Physiol. 234:511-514 (1978); Kamm et al., Preclinical and clinical toxicology of selected retinoids. In “The Retinoids” Vol. 2, pp. 287-326 Academic Press, New York (1984); Nieman and Obbink, Vitam. Horm. 12:69-99 (1954). The roles and regulation of vitamin A and retinoids within a biological system have also been shown to differ. For example, the importance of vitamin A has been shown to be critical in embryonic development, whereas introduction of exogenous retinoic acid suggests teratogenic effects in almost every developing tissue or organ system. See, e.g., Shenfelt, Teratology 5:103-118 (1972); Osmond, et al., Development 113:1405-1417 (1991), Hofmann and Eichele, Retinoids in development. In “The Retinoids: Biology, Chemistry, and Medicine,” 2nd ed. pp. 387-441 Raven Press, New York (1994); Wood, et al., Development 120:2279-2285 (1994), Avantaggiato, et al., Dev. Biol. 175:347-357 (1996); Zhang, et al., Dev. Dynam. 206:73-86 (1996) Therefore, a combination of isotretinoin with dietary supplements that include beta-carotene, a form of vitamin A, would not be an apparent choice of treatment. However, and without being bound to any particular mechanism, because the compounds of Formula (I) and certain vitamins act on different aspects of the visual cycle (as well as the general health of the eye), a combination treatment can act more effectively than either treatment in isolation. By way of example only, isotretinoin and vitamin A may serve different functions. Isotretinoin may act to reduce the production of all-trans-retinal while vitamin A may play a role in the development of the retinal photoreceptor.

The use of certain minerals has also been shown to provide benefit for patients with macular degenerations and dystrophies. See, e.g., Arch. Opthalmol., 119: 1417-36 (2001). By way of example only, suitable minerals that could be used in combination with at least one compound having the structure of Formula (I) include copper-containing minerals, such as cupric oxide (by way of example only); zinc-containing minerals, such as zinc oxide (by way of example only); and selenium-containing compounds.

The compounds of Formula (I) in combination with certain minerals as a second agent can be used to provide benefit to patients suffering from or susceptible to various macular degenerations and dystrophies, including but not limited to dry-form age-related macular degeneration and Stargardt Disease. That is, we consider compounds of Formula (I) in combination with certain minerals to be capable of providing at least some of the following benefits to such human patients: reduction in the amount of all-trans-retinal, reduction in the formation of A2E, reduction in the formation of lipofuscin, reduction in the formation of drusen, and reduction in light sensitivity.

The use of anti-oxidants has been shown to provide benefit for patients with macular degenerations and dystrophies. See, e.g., Arch. Opthalmol., 119: 1417-36 (2001); Sparrow, et al., J. Biol. Chem., 278:18207-13 (2003). By way of example only, suitable anti-oxidants that could be used in combination with at least one compound having the structure of Formula (I) recited herein include coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

The compounds of Formula (I) in combination with certain anti-oxidants as a second agent can be used to provide benefit to patients suffering from or susceptible to various macular degenerations and dystrophies, including but not limited to dry-form age-related macular degeneration and Stargardt Disease. That is, we consider compounds of Formula (I) in combination with certain anti-oxidants to be capable of providing at least some of the following benefits to such human patients: reduction in the amount of all-trans-retinal, reduction in the formation of A2E, reduction in the formation of lipofuscin, reduction in the formation of drusen, and reduction in light sensitivity.

The use of certain negatively-charged phospholipids has also been shown to provide benefit for patients with macular degenerations and dystrophies. See, e.g., Shaban & Richter, Biol. Chem., 383:537-45 (2002); Shaban, et al., Exp. Eye Res., 75:99-108 (2002). By way of example only, suitable negatively charged phospholipids that could be used in combination with at least one compound having the structure of Formula (I) recited herein include lutein, zeaxanthin, cardiolipin and phosphatidylglycerol.

The compounds of Formula (I) in combination with certain negatively-charged phospholipids as a second agent can be used to provide benefit to patients suffering from or susceptible to various macular degenerations and dystrophies, including but not limited to dry-form age-related macular degeneration and Stargardt Disease. That is, we consider compounds of Formula (I) in combination with certain negatively-charged phospholipids to be capable of providing at least some of the following benefits to such human patients: reduction in the amount of all-trans-retinal, reduction in the formation of A2E, reduction in the formation of lipofuscin, reduction in the formation of drusen, and reduction in light sensitivity.

Suitable nitric oxide (NO) inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)). In addition, statins can serve as suitable nitric oxide inducers, include statins, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

The compounds of Formula (I) in combination with suitable nitric oxide as a second agent can be used to provide benefit to patients suffering from or susceptible to various macular degenerations and dystrophies, including but not limited to dry-form age-related macular degeneration and Stargardt Disease. That is, we consider compounds of Formula (I) in combination with suitable nitric oxide to be capable of providing at least some of the following benefits to such human patients: reduction in the amount of all-trans-retinal, reduction in the formation of A2E, reduction in the formation of lipofuscin, reduction in the formation of drusen, and reduction in light sensitivity.

Suitable anti-inflammatory agents with Compounds of Formula (I) recited herein may be used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, Celebrex™, or Vioxx™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

The compounds of Formula (I) in combination with suitable anti-inflammatory agents as a second agent can be used to provide benefit to patients suffering from or susceptible to various macular degenerations and dystrophies, including but not limited to dry-form age-related macular degeneration and Stargardt Disease. That is, we consider compounds of Formula (I) in combination with suitable anti-inflammatory agents to be capable of providing at least some of the following benefits to such human patients: reduction in the amount of all-trans-retinal, reduction in the formation of A2E, reduction in the formation of lipofuscin, reduction in the formation of drusen, and reduction in light sensitivity.

The compounds of Formula (I) in combination with suitable isomers of 13-cis-retinoic acid and their ester and amide derivatives (including by way of example only, all-trans retinoic acid, also known as tretinoin, and fenretinide, respectively) as a second agent can also be used to provide benefit to patients suffering from or susceptible to various macular degenerations and dystrophies, including but not limited to dry-form age-related macular degeneration and Stargardt Disease. That is, we consider compounds of Formula (I) in combination with suitable isomers of 13-cis-retinoic acid and their ester and amide derivatives (including by way of example only, all-trans retinoic acid, also known as tretinoin, and fenretinide, respectively) to be capable of providing at least some of the following benefits to such human patients: reduction in the amount of all-trans-retinal, reduction in the formation of A2E, reduction in the formation of lipofuscin, reduction in the formation of drusen, and reduction in light sensitivity. In addition, such isomers and their derivatives may act in synergy with the compounds of Formula (I), thus allowing administration of lesser amounts of the agent with less desired side effects and/or toxicities.

By way of example only, treatment of compounds of Formula (I) can be administered before, during or after administration of vitamins A and C as a second agent. By way of example only, treatment of compounds of Formula (I) can be administered before, during or after administration of vitamins A and E as a second agent. By way of example only, treatment of compounds of Formula (I) can be administered before, during and/or after administration of vitamins E and C as a second agent. Further options envisioned include multiple administrations of either agent in combination with a single or multiple administrations of the other agent.

By way of example only, treatment of compounds of Formula (I) can be administered before, during and/or after administration of certain minerals as a second agent. By way of example only, treatment of compounds of Formula (I) can be administered before, during and/or after administration of certain anti-oxidants as a second agent. By way of example only, treatment of compounds of Formula (I) can be administered before, during and/or after administration of certain negatively-charged phospholipids as a second agent. By way of example only, treatment of compounds of Formula (I) can be administered before, during and/or after administration of suitable nitric oxide inducers as a second agent. By way of example only, treatment of compounds of Formula (I) can be administered before, during and/or after administration of suitable anti-inflammatory agents as a second agent. Further options envisioned include multiple administrations of and of the first agents in combination with a single or multiple administrations of the second agent.

An additional second agent is DHA, or docosahexaenoic acid, which has been considered a dietary supplementation to improve macular function in patients with Stargardt macular dystrophy and Stargardt-like macular dystrophy. DHA is a fatty acid that is essential for normal brain and eye development. It is normally found in the diet, but not in large amounts. A mutation in the gene, ELOVL4 (elongation of the very long chain fatty acid-4), has been found in individuals with Stargardt-like macular dystrophy. Supplements may help prevent or slow the progression of some eye diseases. Doses of DHA may range from 200-4000 mg/day.

The Visual Cycle. The vertebrate retina contains two types of photoreceptor cells. Rods are specialized for vision under low light conditions. Cones are less sensitive, provide vision at high temporal and spatial resolutions, and afford color perception. Under daylight conditions, the rod response is saturated and vision is mediated entirely by cones. Both cell types contain a structure called the outer segment comprising a stack of membranous discs. The reactions of visual transduction take place on the surfaces of these discs. The first step in vision is absorption of a photon by an opsin-pigment molecule, which involves 11-cis to all-trans isomerization of the retinal chromophore. Before light sensitivity can be regained, the resulting all-trans-retinal must dissociate from the opsin apoprotein and isomerize to 11-cis-retinal.

Macular or Retinal Degenerations and Dystrophies. Macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type. In “dry” macular degeneration, the deterioration of the retina is associated with the formation of small yellow deposits, known as drusen, under the macula. This phenomena leads to a thinning and drying out of the macula. The location and amount of thinning in the retina caused by the drusen directly correlates to the amount of central vision loss. Degeneration of the pigmented layer of the retina and photoreceptors overlying drusen become atrophic and can cause a slow loss of central vision.

Stargardt Disease is a macular dystrophy that manifests as a recessive form of macular degeneration with an onset during childhood. See e.g., Allikmets et al., Science, 277:1805-07 (1997); Lewis et al., Am. J. Hum. Genet., 64:422-34 (1999); Stone et al., Nature Genetics, 20:328-29 (1998); Allikmets, Am. J. Hum. Gen., 67:793-799 (2000); Klevering, et al, Opthalmology, 111:546-553 (2004). Stargardt Disease is characterized clinically by progressive loss of central vision and progressive atrophy of the RPE overlying the macula. Mutations in the human ABCR gene for RmP are responsible for Stargardt Disease. Early in the disease course, patients show delayed dark adaptation but otherwise normal rod function. Histologically, Stargardt Disease is associated with deposition of lipofuscin pigment granules in RPE cells.

Besides Stargardt Disease, mutations in ABCR have been implicated in recessive retinitis pigmentosa, see, e.g., Cremers et al., Hum. Mol. Genet., 7:355-62 (1998), recessive cone-rod dystrophy, see id., and non-exudative age-related macular degeneration (AMD), see e.g., Allikmets et al., Science, 277:1805-07 (1997); Lewis et al., Am. J. Hum. Genet., 64:422-34 (1999), although the prevalence of ABCR mutations in AMD is still uncertain. See Stone et al., Nature Genetics, 20:328-29 (1998); Allikmets, Am. J. Hum. Gen., 67:793-799 (2000); Klevering, et al, Opthalmology, 111:546-553 (2004). Similar to Stargardt Disease, these diseases are associated with delayed rod dark-adaptation. See Steinmetz et al., Brit. J. Ophthalm., 77:549-54 (1993). Lipofuscin deposition in RPE cells is also seen prominently in AMD, see Kliffen et al., Microsc. Res. Tech., 36:106-22 (1997) and some cases of retinitis pigmentosa. See Bergsma et al., Nature, 265:62-67 (1977).

Chemical Terminology

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 10 carbon atoms (whenever it appears herein, a numerical range such as “1 to 10” refers to each integer in the given range; e.g., “1 to 40 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 5 carbon atoms. The alkyl group of the compounds described herein may be designated as “C1-C4 alkyl” or similar designations. By way of example only, “C1-C4 alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkylamine” refers to the —N(alkyl)xHy group, where x and y are selected from the group x=1, y=1 and x=2, y=0. When x=2, the alkyl groups, taken together, can optionally form a cyclic ring system.

The term “alkenyl” refers to a type of alkyl group in which the first two atoms of the alkyl group form a double bond that is not part of an aromatic group. That is, an alkenyl group begins with the atoms —C(R)═C—R, wherein R refers to the remaining portions of the alkenyl group, which may be the same or different. Non-limiting examples of an alkenyl group include —CH═CH, —C(CH3)═CH, —CH═CCH3 and —C(CH3)═CCH3. The alkenyl moiety may be branched, straight chain, or cyclic (in which case, it would also be known as a “cycloalkenyl” group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH3 and —C≡CCH2CH3. The “R” portion of the alkynyl moiety may be branched, straight chain, or cyclic.

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

The term “aromatic” or “aryl” refers to an aromatic group which has at least one ring having a conjugated pi electron system and includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

A “cyano” group refers to a —CN group.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

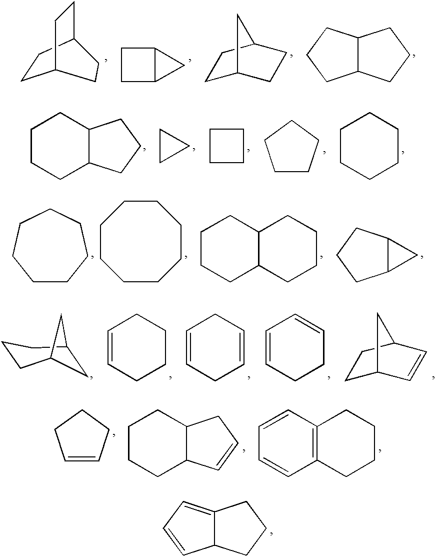

and the like.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

The term “halo” or, alternatively, “halogen” means fluoro, chloro, bromo or iodo. Preferred halo groups are fluoro, chloro and bromo.

The terms “haloalkyl,” “haloalkenyl,” “haloalkynyl” and “haloalkoxy” include alkyl, alkenyl, alkynyl and alkoxy structures, that are substituted with one or more halo groups or with combinations thereof. The terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

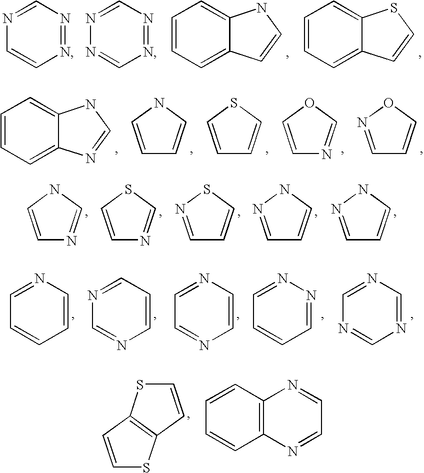

and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

A “heteroalicyclic” group refers to a cycloalkyl group that includes at least one heteroatom selected from nitrogen, oxygen and sulfur. The radicals may be fused with an aryl or heteroaryl. Illustrative examples of heterocycloalkyl groups include:

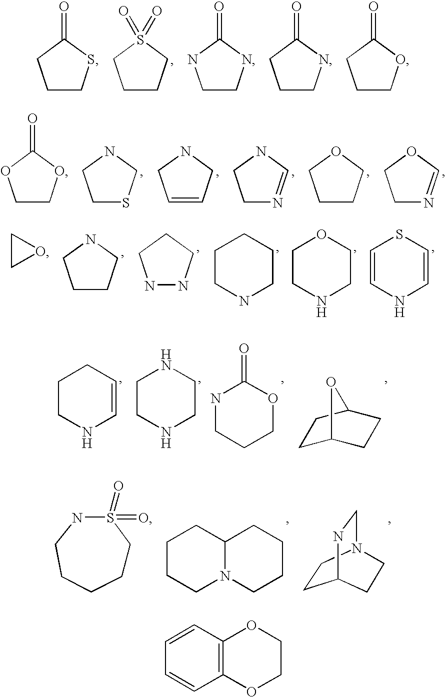

The term heteroalicyclic also includes all ring forms of the carbohydrates, including but not limited to the monosaccharides, the disaccharides and the oligosaccharides.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.
An “isocyanato” group refers to a —NCO group.
An “isothiocyanato” group refers to a —NCS group.
A “mercaptyl” group refers to a (alkyl)S— group.

The terms “nucleophile” and “electrophile” as used herein have their usual meanings familiar to synthetic and/or physical organic chemistry. Carbon electrophiles typically comprise one or more alkyl, alkenyl, alkynyl or aromatic (sp3, sp2, or sp hybridized) carbon atoms substituted with any atom or group having a Pauling electronegativity greater than that of carbon itself. Examples of carbon electrophiles include but are not limited to carbonyls (aldehydes, ketones, esters, amides), oximes, hydrazones, epoxides, aziridines, alkyl-, alkenyl-, and aryl halides, acyls, sulfonates (aryl, alkyl and the like). Other examples of carbon electrophiles include unsaturated carbon atoms electronically conjugated with electron withdrawing groups, examples being the 6-carbon in alpha-unsaturated ketones or carbon atoms in fluorine substituted aryl groups. Methods of generating carbon electrophiles, especially in ways which yield precisely controlled products, are known to those skilled in the art of organic synthesis.

The relative disposition of aromatic substituents (ortho, meta, and para) imparts distinctive chemistry for such stereoisomers and is well recognized within the field of aromatic chemistry. Para- and meta-substitutional patterns project the two substituents into different orientations. Ortho-disposed substituents are oriented at 60° with respect to one another; meta-disposed substituents are oriented at 120° with respect to one another; para-disposed substituents are oriented at 180° with respect to one another.

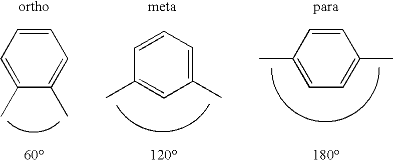

Relative dispositions of substituents, viz, ortho, meta, para, also affect the electronic properties of the substituents. Without being bound to any particular type or level of theory, it is known that ortho- and para-disposed substituents electronically affect one another to a greater degree than do corresponding meta-disposed substituents. Meta-disubstituted aromatics are often synthesized using different routes than are the corresponding ortho and para-disubstituted aromatics.

The term “moiety” refers to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

A “sulfinyl” group refers to a —S(═O)—R, where R is selected from the group consisting of alkyl, cycloalkyl, aryl, heteroaryl (bonded through a ring carbon) and heteroalicyclic (bonded through a ring carbon)

A “sulfonyl” group refers to a —S(═O)₂—R, where R is selected from the group consisting of alkyl, cycloalkyl, aryl, heteroaryl (bonded through a ring carbon) and heteroalicyclic (bonded through a ring carbon)

A “thiocyanato” group refers to a —CNS group.

The term “optionally substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. The protecting groups that may form the protective derivatives of the above substituents are known to those of skill in the art and may be found in references such as Greene and Wuts, above.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (I), as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Pharmaceutical Compositions

In another aspect are pharmaceutical compositions comprising a compound of Formula (I), as described herein, and a pharmaceutically acceptable diluent, excipient, or carrier.

The term “pharmaceutical composition” refers to a mixture of a compound of Formula (I) in combination with a second agent recited herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “carrier” refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (providing pH control) are utilized as diluents in the art. One commonly used buffered solution is phosphate buffered saline. It is a buffer found naturally in the blood system.

The term “physiologically acceptable” refers to a material, such as a carrier or diluent, that does not abrogate the biological activity or properties of the compound, and is nontoxic.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound of Formula (I) in combination with a second agent recited herein with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts may also be obtained by reacting a compound of Formula (I) in combination with a second agent recited herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art

A “metabolite” of a compound disclosed herein is a derivative of that compound that is formed when the compound is metabolized. The term “active metabolite” refers to a biologically active derivative of a compound that is formed when the compound is metabolized. The term “metabolized” refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of Formula (I) in combination with a second agent recited herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The compounds described herein can be administered to a human patient per se, or in pharmaceutical compositions where they are mixed with other active ingredients, as in combination therapy, or suitable carrier(s) or excipient(s). Techniques for formulation and administration of the compounds of the instant application may be found in “Remington: The Science and Practice of Pharmacy,” 20th ed. (2000).

Routes of Administration

Suitable routes of administration may, for example, include oral, rectal, transmucosal, pulmonary, ophthalmic or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into an organ, often in a depot or sustained release formulation. Furthermore, one may administer the drug in a targeted drug delivery system, for example, in a liposome coated with organ-specific antibody. The liposomes will be targeted to and taken up selectively by the organ.

Composition/Formulation

Pharmaceutical compositions comprising a compound of Formula (I) and/or a second agent recited herein may be manufactured in a manner that is itself known, e.g., by means of conventional mixing, dissolving, granulating, dragee-making, levigating, emulsifying, encapsulating, entrapping or compression processes.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

The compounds of Formula (I) and/or a second agent recited herein can be administered in a variety of ways, including all forms of local delivery to the eye. Additionally, the compounds of Formula (I) and/or a second agent recited herein can be administered systemically, such as orally or intravenously. The compounds of Formula (I) and/or a second agent recited herein can be administered topically to the eye and can be formulated into a variety of topically administrable ophthalmic compositions, such as solutions, suspensions, gels or ointments. Thus, “ophthalmic administration” encompasses, but is not limited to, intraocular injection, subretinal injection, intravitreal injection, periocular administration, subconjuctival injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the fornix).

Administration of a composition to the eye generally results in direct contact of the agents with the cornea, through which at least a portion of the administered agents pass. Often, the composition has an effective residence time in the eye of about 2 to about 24 hours, more typically about 4 to about 24 hours and most typically about 6 to about 24 hours.

A composition comprising a compound of Formula (I) and/or a second agent recited herein can illustratively take the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition may include a gel formulation. In other embodiments, the liquid composition is aqueous. Alternatively, the composition can take the form of an ointment.

Useful compositions can be an aqueous solution, suspension or solution/suspension, which can be presented in the form of eye drops. A desired dosage can be administered via a known number of drops into the eye. For example, for a drop volume of 25 μl, administration of 1-6 drops will deliver 25-150 μl of the composition. Aqueous compositions typically contain from about 0.01% to about 50%, more typically about 0.1% to about 20%, still more typically about 0.2% to about 10%, and most typically about 0.5% to about 5%, weight/volume of a compound of Formula (I) and/or a second agent recited herein.

Typically, aqueous compositions have ophthalmically acceptable pH and osmolality. “Ophthalmically acceptable” with respect to a formulation, composition or ingredient typically means having no persistent detrimental effect on the treated eye or the functioning thereof, or on the general health of the subject being treated.

Transient effects such as minor irritation or a “stinging” sensation are common with topical ophthalmic administration of agents and consistent with the formulation, composition or ingredient in question being “ophthalmically acceptable.”

Useful aqueous suspension can also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions can also comprise an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions may also include ophthalmically acceptable solubilizing agent to aid in the solubility of a compound of Formula (I) and/or a second agent recited herein. The term “solubilizing agent” generally includes agents that result in formation of a micellar solution or a true solution of the agent. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

Useful compositions may also include one or more ophthalmically acceptable pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

Useful compositions may also include one or more ophthalmically acceptable salts in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful compositions may also include one or more ophthalmically acceptable preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions may include one or more ophthalmically acceptable surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions may include one or more antioxidants to enhance chemical stability where required. Suitable antioxidants include, by way of example only, ascorbic acid and sodium metabisulfite.

Aqueous suspension compositions can be packaged in single-dose non-reclosable containers. Alternatively, multiple-dose reclosable containers can be used, in which case it is typical to include a preservative in the composition.

The ophthalmic composition may also take the form of a solid article that can be inserted between the eye and eyelid or in the conjunctival sac, where it releases the agent. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable.

For intravenous injections, compounds of Formula (I) and/or a second agent recited herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

One useful formulation for solubilizing higher quantities of the compounds of Formula (I) are, by way of example only, positively, negatively or neutrally charged phospholipids, or bile salt/phosphatidylcholine mixed lipid aggregate systems, such as those described in Li, C. Y., et al., Pharm. Res. 13:907-913 (1996). An additional formulation that can be used for the same purpose with compounds having the structure of Formula (I) involves use of a solvent comprising an alcohol, such as ethanol, in combination with an alkoxylated caster oil. See, e.g., U.S. Patent Publication Number 2002/0183394. Or, alternatively, a formulation comprising compounds of Formula (I) in an emulsion composed of a lipoid dispersed in an aqueous phase, a stabilizing amount of a non-ionic surfactant, optionally a solvent, and optionally an isotonic agent. See id.

For oral administration, compounds of Formula (I) and/or a second agent recited herein can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated. Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner.

Another useful formulation for administration of compounds having the structure of Formula (I) and/or a second agent recited herein employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252. Such patches may be constructed for continuous, pulsatile, or on demand delivery of pharmaceutical agents. Still further, transdermal delivery of the compounds of Formula (I) and/or a second agent recited herein can be accomplished by means of iontophoretic patches and the like. Transdermal patches can provide controlled delivery of the compounds. The rate of absorption can be slowed by using rate-controlling membranes or by trapping the compound within a polymer matrix or gel. Conversely, absorption enhancers can be used to increase absorption. Formulations suitable for transdermal administration can be presented as discrete patches and can be lipophilic emulsions or buffered, aqueous solutions, dissolved and/or dispersed in a polymer or an adhesive. Transdermal patches may be placed over different portions of the patient's body, including over the eye.

Additional iontophoretic devices that can be used for ocular administration of compounds having the structure of Formula (I) and/or a second agent recited herein are the Eyegate applicator, created and patented by Optis France S.A., and the Ocuphor™ Ocular iontophoresis system developed Iomed, Inc.

For administration by inhalation, the compounds of Formula (I) and/or a second agent recited herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as rectal gels, rectal foam, rectal aerosols, suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Injectable depot forms may be made by forming microencapsule matrices of the compound of Formula (I) and/or a second agent recited herein in biodegradable polymers. Depending upon the ratio of drug to polymer and the nature of the particular polymer employed, the rate of drug release can be controlled. Depot injectable formulations may be also prepared by entrapping the drug in liposomes or microemulsions. By way of example only, posterior juxtascleral depots may be used as a mode of administration for compounds having the structure of Formula (I) and/or a second agent recited herein. The sclera is a thin avascular layer, comprised of highly ordered collagen network surrounding most of vertebrate eye. Since the sclera is avascular it can be utilized as a natural storage depot from which injected material cannot rapidly removed or cleared from the eye. The formulation used for administration of the compound into the scleral layer of the eye can be any form suitable for application into the sclera by injection through a cannula with small diameter suitable for injection into the scleral layer. Examples for injectable application forms are solutions, suspensions or colloidal suspensions.

A pharmaceutical carrier for the hydrophobic compounds of Formula (I) is a cosolvent system comprising benzyl alcohol, a nonpolar surfactant, a water-miscible organic polymer, and an aqueous phase. The cosolvent system may be a 10% ethanol, 10% polyethylene glycol 300, 10% polyethylene glycol 40 castor oil (PEG-40 castor oil) with 70% aqueous solution. This cosolvent system dissolves hydrophobic compounds well, and itself produces low toxicity upon systemic administration. Naturally, the proportions of a cosolvent system may be varied considerably without destroying its solubility and toxicity characteristics. Furthermore, the identity of the cosolvent components may be varied: for example, other low-toxicity nonpolar surfactants may be used instead of PEG-40 castor oil, the fraction size of polyethylene glycol 300 may be varied; other biocompatible polymers may replace polyethylene glycol, e.g., polyvinyl pyrrolidone; and other sugars or polysaccharides maybe included in the aqueous solution.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

All of the formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

Many of the compounds of Formula (I) in combination with a second agent recited herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Treatment Methods, Dosages and Combination Therapies

The term “mammal” means all mammals including humans. Mammals include, by way of example only, humans, non-human primates, cows, dogs, cats, goats, sheep, pigs, rats, mice and rabbits.

The term “effective amount” as used herein refers to that amount of the compound being administered which will relieve to some extent one or more of the symptoms of the disease, condition or disorder being treated.

The compositions containing the compound(s) described herein can be administered for prophylactic and/or therapeutic treatments. The term “treating” is used to refer to either prophylactic and/or therapeutic treatments. In therapeutic applications, the compositions are administered to a patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial).

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a patient, amounts effective for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be temporarily suspended for a certain length of time (i.e., a “drug holiday”).

Once improvement of the patient's status has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day.

In certain instances, it may be appropriate to administer at least one of the compounds described herein (or a pharmaceutically acceptable salt, ester, amide, prodrug, or solvate) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for macular degeneration involving administration of one of the compounds described herein, increased therapeutic benefit may result by also providing the patient with another therapeutic agents or therapies for macular degeneration. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of at least one compound of Formula (I) and a second agent recited herein with vitamins, minerals, nitric oxide inducers, negatively charged phospholipids, anti-oxidants, minerals, and anti-inflammatory agents. In several instances, suitable combination agents may fall within multiple categories (by way of example only, lutein is both an anti-oxidant and a negatively charged phospholipid).

The compounds of Formula (I) and a second agent recited herein may also be used in combination with procedures that may provide additional or synergistic benefit to the patient, including, by way of example only, the use of extracorporeal rheopheresis (also known as membrane differential filtration), the use of implantable miniature telescopes, laser photocoagulation of drusen, and microstimulation therapy. Further, the compounds of Formula (I) and a second agent recited herein may also be administered with additional agents that may provide benefit to the patient, including by way of example only anacortave acetate and cyclosporin A.

The use of certain vitamins has been shown to provide benefit for patients with macular degenerations and dystrophies. In particular, vitamins A, C and E seem to have an effect in the healthy maintenance of the eye. Vitamin A has a role in the formation of the retinal photoreceptor pigments and lack of it can lead to a decrease in night vision. See, e.g., Brown, et al. A high concentration of vitamin C can be found in the aqueous humour which suggests its important role in maintenance of the eye lens. See, e.g., Taylor, et al., Curr. Eye Res., 10:751-9 (1991). Vitamin C also has a role in reducing the development of cataracts and protecting the retina from light damage. See, e.g., Tso, et al., Curr. Eye Res., 3:166-74 (1984); Robertson, et al., Ann. NY Acad. Sci., 570:372-82 (1989). Vitamin E has been implicated in reducing the risk of cortical, nuclear and mixed cataract types. See, e.g., Leske, et al., Arch. Opthalmol., 109:244-51 (1991). Examples of suitable vitamins could be used in combination with at least one compound having the structure of Formula (I) and a second agent recited herein to provide benefit to patients suffering from or susceptible to various macular degenerations and dystrophies, including but not limited to dry-form age-related macular degeneration and Stargardt Disease.

The use of certain minerals has also been shown to provide benefit for patients with macular degenerations and dystrophies. See, e.g., Arch. Opthalmol., 119: 1417-36 (2001). Examples of suitable minerals that could be used in combination with at least one compound having the structure of Formula (I) include copper-containing minerals, such as cupric oxide (by way of example only); zinc-containing minerals, such as zinc oxide (by way of example only); and selenium-containing compounds. Examples of suitable minerals could be used in combination with at least one compound having the structure of Formula (I) and a second agent recited herein to provide benefit to patients suffering from or susceptible to various macular degenerations and dystrophies, including but not limited to dry-form age-related macular degeneration and Stargardt Disease.

The use of anti-oxidants has been shown to provide benefit for patients with macular degenerations and dystrophies. See, e.g., Arch. Opthalmol., 119: 1417-36 (2001); Sparrow, et al., J. Biol. Chem., 278:18207-13 (2003). Examples of suitable anti-oxidants that could be used in combination with at least one compound having the structure of Formula (I) and a second agent recited herein to include as a third agent coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), or bilberry extract.

The use of certain negatively-charged phospholipids has also been shown to provide benefit for patients with macular degenerations and dystrophies. See, e.g., Shaban & Richter, Biol. Chem., 383:537-45 (2002); Shaban, et al., Exp. Eye Res., 75:99-108 (2002). Examples of suitable negatively charged phospholipids that could be used in combination with at least one compound having the structure of Formula (I) and a second agent recited herein to include as a third agent lutein, zeaxanthin, cardiolipin or phosphatidylglycerol.

Suitable nitric oxide (NO) inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)). In addition, statins can serve as suitable nitric oxide inducers, include statins, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin. Examples of suitable nitric oxide inducers recited herein could be used in combination as a third agent will at least one compound having the structure of Formula (I) and a second agent recited herein.

Suitable anti-inflammatory agents with which the compounds of Formula (I) and a second agent recited herein may be used to include as a third agent aspirin or other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, Celebrex™, or Vioxx™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

By way of example only, an exemplary order of administration of the compounds could be as follows: the first agent being compounds of Formula (I), the second agent being certain vitamins and the third agent being minerals. Another exemplary order of administration of the compounds could be as follows: the first agent being compounds of Formula (I), the second agent being certain vitamins and the third agent being certain anti-oxidants. Another exemplary order of administration of the compounds could be as follows: the first agent being compounds of Formula (I), the second agent being certain vitamins and the third agent being certain negatively-charged phospholipids. Another exemplary order of administration of the compounds could be as follows: the first agent being compounds of Formula (I), the second agent being certain vitamins and the third agent being suitable nitric oxide inducers. Another exemplary order of administration of the compounds could be as follows: the first agent being compounds of Formula (I), the second agent being certain vitamins and the third agent being suitable anti-inflammatory agents.

By way of example only, an exemplary order of administration of the compounds could be as follows: the first agent being compounds of Formula (I), the second agent being certain minerals and the third agent being certain anti-oxidants. Another exemplary order of administration of the compounds could be as follows: the first agent being compounds of Formula (I), the second agent being certain minerals and the third agent being certain negatively-charged phospholipids. Another exemplary order of administration of the compounds could be as follows: the first agent being compounds of Formula (I), the second agent being certain minerals and the third agent being suitable nitric oxide inducers. Another exemplary order of administration of the compounds could be as follows: the first agent being compounds of Formula (I), the second agent being certain minerals and the third agent being suitable anti-inflammatory agents.

By way of example only, an exemplary order of administration of the compounds could be as follows: the first agent being compounds of Formula (I), the second agent being certain anti-oxidants and the third agent being certain negatively-charged phospholipids. Another exemplary order of administration of the compounds could be as follows: the first agent being compounds of Formula (I), the second agent being certain anti-oxidants and the third agent being suitable nitric oxide inducers. Another exemplary order of administration of the compounds could be as follows: the first agent being compounds of Formula (I), the second agent being certain anti-oxidants and the third agent being suitable anti-inflammatory agents.

By way of example only, an exemplary order of administration of the compounds could be as follows: the first agent being compounds of Formula (I), the second agent being certain negatively-charged phospholipids and the third agent being suitable nitric oxide inducers. Another exemplary order of administration of the compounds could be as follows: the first agent being compounds of Formula (I), the second agent being certain negatively-charged phospholipids and the third agent being suitable anti-inflammatory agents.

By way of example only, an exemplary order of administration of the compounds could be as follows: the first agent being compounds of Formula (I), the second agent being suitable nitric oxide inducers and the third agent being suitable anti-inflammatory agents.

Exemplary variations on the timing of administration of these agents by way of example only and upon a doctor's discretion, wherein agents are administered one after the other in succession with all the possible combination of arrangements to choose from; one agent is administered at the same time as another and before administration of the last agent; one agent is administered at the same time as another and after administration of the last agent; all three agents are administered at the same time; and specific agents are administered at multiple doses.

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations may not be limited to the use of only two agents; we envision the use of multiple therapeutic combinations. By way of example only, a compound having the structure of Formula (I) and a second agent recited herein may be provided with at least one additional antioxidant and at least one negatively charged phospholipid; or a compound having the structure of Formula (I) and a second agent recited herein may be provided with at least one additional antioxidant and at least one inducer of nitric oxide production; or a compound having the structure of Formula (I) and a second agent recited herein may be provided with at least one inducer of nitric oxide productions and at least one negatively charged phospholipid; and so forth.

Further combinations that may be used to provide benefit to an individual include the use of genetic testing to determine whether that individual is a carrier of a mutant gene that is known to be correlated with certain ophthalmic conditions. By way of example only, defects in the human ABCR gene are thought to be associated with five distinct retinal phenotypes including Stargardt disease, cone-rod dystrophy, age-related macular degeneration and retinitis pigmentosa. See e.g., Allikmets et al., Science, 277:1805-07 (1997); Lewis et al., Am. J. Hum. Genet., 64:422-34 (1999); Stone et al., Nature Genetics, 20:328-29 (1998); Allikmets, Am. J. Hum. Gen., 67:793-799 (2000); Klevering, et al, Opthalmology, 111:546-553 (2004). Such patients would be expected to find therapeutic and/or prophylactic benefit in the methods described herein.

Synthesis of the Compounds of Formula (I)

Compounds of Formula (I) may be synthesized using standard synthetic techniques known to those of skill in the art or using methods known in the art in combination with methods described herein. See, e.g., U.S. Patent Application Publication 2004/0102650; Um, S. J., et al., Chem. Pharm. Bull., 52:501-506 (2004). In addition, several of the compounds of Formula (I), such as fenretinide, may be purchased from various commercial suppliers. As a further guide the following synthetic methods may also be utilized.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof.” Precursor functional groups are shown as electrophilic groups and nucleophilic groups. The functional group on the organic substance may be attached directly, or attached via any useful spacer or linker as defined below.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, e.g, oxygen or nitrogen.

Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd0-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

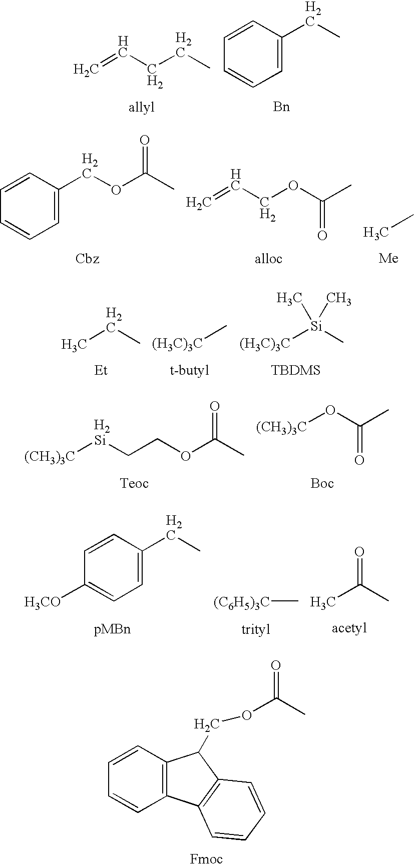

Other protecting groups are described in Greene and Wuts, Protective Groups in Organic Synthesis, 3rd Ed., John Wiley & Sons, New York, N.Y., 1999, which is incorporated herein by reference in its entirety.

### ILLUSTRATIVE EXAMPLES

The following examples provide illustrative methods for testing the effectiveness and safety of the compounds of Formula (I). These examples are provided for illustrative purposes only and not to limit the scope of the claims provided herein.

Human Studies

Detection of Macular or Retinal Degeneration. Identification of abnormal blood vessels in the eye can be done with an angiogram. This identification can help determine which patients are candidates for the use of a candidate substance or other treatment method to hinder or prevent further vision loss. Angiograms can also be useful for follow-up of treatment as well as for future evaluation of any new vessel growth.

A fluorescein angiogram (fluorescein angiography, fluorescein angioscopy) is a technique for the visualization of choroidal and retinal circulation at the back of the eye. Fluorescein dye is injected intravenously followed by multiframe photography (angiography) or opthalmoscopic evaluation (angioscopy). Fluorescein angiograms are used in the evaluation of a wide range of retinal and choroidal diseases through the analysis of leakage or possible damage to the blood vessels that feed the retina. It has also been used to evaluate abnormalities of the optic nerve and iris by Berkow et al. (1984).

Similarly, angiograms using indocyanine green can be used for the visualization circulation at the back of the eye. Wherein fluorescein is more efficient for studying retinal circulation, indocyanine is better for observing the deeper choroidal blood vessel layer. The use of indocyanine angiography is helpful when neovascularization may not be observed with fluorescein dye alone.

Appropriate human doses for compounds having the structure of Formula (I) and/or a second agent recited herein will be determined using a standard dose escalation study. However, some guidance is available from the studies on the use of isotretinoin therapy in the treatment of severe nodular acne and studies on the use of dietary supplementation in patients with age-related macular degeneration. See, e.g., Chang, et al. Can. J. Opthalmol. 38:27-32 (2003); Kaminski, et al., J. Am. Optometric Ass., 64:862-870 (1993).

### Example 1

### Testing for the Efficacy of Compounds of Formula (I) in Combination with a Second Agent to Treat Macular Degeneration

For pre-testing, all human patients undergo a routine ophthalmologic examination including fluorescein angiography, measurement of visual acuity, electrophysiologic parameters and biochemical and rheologic parameters. Inclusion criteria are as follows: visual acuity between 20/160 and 20/32 in at least one eye and signs of ARMD such as drusen, areolar atrophy, pigment clumping, pigment epithelium detachment, or subretinal neovascularization. Patients with any of the following are excluded from the study: dementia; severe cardiac disease; history of malignancy or infection with hepatitis, or Treponema pallidum; and suitability for laser coagulation according to the guidelines of the Macular Photocoagulation Study Group (Arch Opthalmol 1991; 10:1 109-1114). Details from Brunner et al. Retina 2000; 20:483-491.

Fifty human patients diagnosed with macular degeneration, or who have progressive formations of A2E, lipofuscin, or drusen in their eyes are divided into a control group of about 25 patients and an experimental group of 25 patients. Compounds of Formula (I) in combination with a second agent recited herein are administered to the experimental group on a daily basis. A placebo is administered to the control group in the same regime as compounds of Formula (I) in combination with a second agent recited herein are administered to the experimental group.

Administration of Formula (I) in combination with a second agent recited herein or placebo to a patient can be either orally or parenterally administered at amounts effective to inhibit the development or reoccurrence of macular degeneration. Effective dosage amounts may be in the range of from about 0.1 mg/kg per day to 1.0 mg/kg per day of isotretinoin with 600 mg vitamin C, 450 mg vitamin E, 30,000 IU vitamin A, 90 mg zinc and 2.5 mg copper for 15 to 20 weeks.

Methods for measuring progression of macular degeneration in both control and experimental groups include taking fundus photographs and fluorescein angiograms at baseline, three, six, nine and twelve months at follow-up visits. Documentation of morphologic changes may include changes in (a) drusen size, character, and distribution (b) development and progression of choroidal neovascularization and (c) other interval fundus changes or abnormalities.

Another method of measuring progression of macular degeneration in both control and experimental groups include acuity tests, Amsler Grid Test, and color testing.

Another method of measuring progression of macular degeneration in both control and experimental groups may be the best corrected visual acuity as measured by Early Treatment Diabetic Retinopathy Study (ETDRS) charts (Lighthouse, Long Island, N.Y.) using line assessment and the forced choice method (Ferris et al. Am J Opthalmol 1982; 94:91-96). Visual acuity may be recorded in logMAR. The change of one line on the ETDRS chart is equivalent to 0.1 logMAR.

To assess statistically visual improvement during drug administration, examiners may use the ETDRS (LogMAR) chart and a standardized refraction and visual acuity protocol. Evaluation of the mean ETDRS (LogMAR) best corrected visual acuity (BCVA) from baseline through the available post-treatment interval visits can aid in determining statistical visual improvement.

To assess the ANOVA (analysis of variance between groups) between the control and experimental group, the mean changes in ETDRS (LogMAR) visual acuity from baseline through the available post-treatment interval visits are compared using two-group ANOVA with repeated measures analysis with unstructured covariance using SAS/STAT Software (SAS Institutes Inc, Cary, N.C.).

Toxicity evaluation after the study may include check ups every three months during the subsequent year, every four months the year after and subsequently every six months. Plasma levels of Formula (I) can also be assessed during these visits. The toxicity evaluation includes patients using Formula (I) as well as the patients in the control group.

### Example 2

### Testing for the Efficacy of Compounds of Formula (I) in Combination with a Second Agent to Reduce A2E Production

The same pre-testing, administration and toxicity evaluation protocols are used as in Example 1. One method for measuring progressive formation of A2E in both control and experimental groups includes the use of a confocal scanning laser opthalmoscope. See Bindewald, et al., Am. J. Opthalmol., 137:556-8 (2004). Documentation of morphologic changes may include changes in (a) drusen size, character, and distribution (b) development and progression of choroidal neovascularization and (c) other interval fundus changes or abnormalities.

To assess statistically visual improvement during drug administration, examiners may use the ETDRS (LogMAR) chart and a standardized refraction and visual acuity protocol. Evaluation of the mean ETDRS (LogMAR) best corrected visual acuity (BCVA) from baseline through the available posttreatment interval visits can aid in determining statistical visual improvement.

To assess the ANOVA (analysis of variance between groups) between the control and experimental group, the mean changes in ETDRS (LogMAR) visual acuity from baseline through the available posttreatment interval visits are compared using two-group ANOVA with repeated measures analysis with unstructured covariance using SAS/STAT Software (SAS Institutes Inc, Cary, N.C.).

### Example 3

### Testing for the Efficacy of Compounds of Formula (I) in Combination with a Second Agent to Reduce Lipofuscin Production

The same pre-testing, administration and toxicity evaluation protocols are used as in Example 1. One method for measuring progressive formation of lipofuscin in both control and experimental groups includes the use of a confocal scanning laser opthalmoscope. Documentation of morphologic changes may include changes in (a) drusen size, character, and distribution (b) development and progression of choroidal neovascularization and (c) other interval fundus changes or abnormalities.

To assess statistically visual improvement during drug administration, examiners may use the ETDRS (LogMAR) chart and a standardized refraction and visual acuity protocol. Evaluation of the mean ETDRS (LogMAR) best corrected visual acuity (BCVA) from baseline through the available posttreatment interval visits can aid in determining statistical visual improvement.

To assess the ANOVA (analysis of variance between groups) between the control and experimental group, the mean changes in ETDRS (LogMAR) visual acuity from baseline through the available posttreatment interval visits are compared using two-group ANOVA with repeated measures analysis with unstructured covariance using SAS/STAT Software (SAS Institutes Inc, Cary, N.C.).

### Example 4

### Testing for the Efficacy of Compounds of Formula (I) in Combination with a Second Agent to Reduce Drusen Production

The same pre-testing, administration and toxicity evaluation protocols are used as in Example 1. Methods for measuring progressive formations of drusen in both control and experimental groups include taking fundus photographs and fluorescein angiograms at baseline, three, six, nine and twelve months at follow-up visits. Documentation of morphologic changes may include changes in (a) drusen size, character, and distribution (b) development and progression of choroidal neovascularization and (c) other interval fundus changes or abnormalities.

Another method of measuring progressive formations of drusen in both control and experimental groups include acuity tests, Amsler Grid Test, and color testing.

Another method of measuring progressive formations of drusen in both control and experimental groups may be the best corrected visual acuity as measured by Early Treatment Diabetic Retinopathy Study (ETDRS) charts (Lighthouse, Long Island, N.Y.) using line assessment and the forced choice method (Ferris et al. Am J Opthalmol 1982; 94:91-96). Visual acuity may be recorded in logMAR. The change of one line on the ETDRS chart is equivalent to 0.1 logMAR.

To assess statistically visual improvement during drug administration, examiners may use the ETDRS (LogMAR) chart and a standardized refraction and visual acuity protocol. Evaluation of the mean ETDRS (LogMAR) best corrected visual acuity (BCVA) from baseline through the available posttreatment interval visits can aid in determining statistical visual improvement.

To assess the ANOVA (analysis of variance between groups) between the control and experimental group, the mean changes in ETDRS (LogMAR) visual acuity from baseline through the available posttreatment interval visits are compared using two-group ANOVA with repeated measures analysis with unstructured covariance using SAS/STAT Software (SAS Institutes Inc, Cary, N.C.).

### Example 5

### Dosage of Formula (I) in Combination with a Second Agent for Administration

Human subjects are tested in the manner described in Examples 1-4, but with an additional two arms. In one of the additional arms, groups of subjects are treated with isotretinoin (0.1 mg/kg/day to 1.0 mg/kg/day) and no supplements. In the second additional arm, groups of subjects are treated with isotretinoin (0.1 mg/kg/day to 1.0 mg/kg/day) and supplements with increasing concentrations from 50 mg to about 600 mg vitamin C, 20 IU to about 450 mg vitamin E, 900 IU to about 30,000 IU vitamin A, 10 mg to about 90 mg zinc, and 0.5 mg to about 2.5 mg copper. The benefits of the dosage for administration are assayed as described in Examples 1-4.

### Example 6

### Suitable Pharmaceutically Acceptable Carrier for Administration

Human subjects are tested in the manner described in Examples 1-5, but with an additional four arms. In one of the additional arms, groups of subjects are administered orally with Formula (I) in combination a second agent. In the second additional arm, groups of subjects are intravenously administered with Formula (I) in combination with a second agent. In the third additional arm, groups of subjects are ophthalmically administered with Formula (I) in combination with a second agent. In the fourth additional arm, groups of subjects are administered by injection with Formula (I) in combination with a second agent. In all of these arms, the second agent is administered orally. An effective amount of a second agent comprising an agent selected from the group consisting of an antioxidant, a mineral, an inducer of nitric oxide production, an anti-inflammatory agent, and a negatively charged phospholipid. The benefits of the carrier for administration are assayed as described in Examples 1-5.

### Example 7

### Genetic Testing for Macular Dystrophies

Defects in the human ABCR gene are thought to be associated with five distinct retinal phenotypes including Stargardt Disease, cone-rod dystrophy, age-related macular degeneration and retinitis pigmentosa. See e.g., Allikmets et al., Science, 277:1805-07 (1997); Lewis et al., Am. J. Hum. Genet., 64:422-34 (1999); Stone et al., Nature Genetics, 20:328-29 (1998); Allikmets, Am. J. Hum. Gen., 67:793-799 (2000); Klevering, et al, Opthalmology, 111:546-553 (2004). Patients can be diagnosed as having Stargardt Disease by a number of assays, including but not limited to:

A direct-sequencing mutation detection strategy which can involve sequencing all exons and flanking intron regions of ABCR for sequence mutation(s);

Genomic Southern analysis;

Microarray assays that include all known ABCR variant; and

Analysis by liquid chromatography tandem mass spectrometry coupled with Western analysis.

Fundus photographs, fluorescein angiograms, and scanning laser opthalmoscope imaging along with the history of the patient and his or her family can anticipate and/or confirm diagnosis.

Mice and Rat Studies

The optimal dose of compounds of Formula (I) in combination with a second agent recited herein to block formation of A2E in abcr−/− mice can be determined using a standard dose escalation study. One illustrative approach, utilizing compounds of Formula (I) in combination with a second agent is presented below. However, similar approaches may be utilized for other compounds having the structure of Formula (I) and/or in combination with a second agent.

The effects of Formula (I) in combination with a second agent on all-trans-retinal in retinas from light-adapted mice would preferably be determined at doses that bracket the human therapeutic dose. The preferred method includes treating mice with a single morning intraperitoneal dose. An increased frequency of injections may be required to maintain reduced levels of all-trans-retinal in the retina throughout the day.

ABCR Knockout Mice. ABCR encodes rim protein (RmP), an ATP-binding cassette (ABC) transporter in the outer-segment discs of rod and cone photoreceptors. The transported substrate for RmP is unknown. Mice generated with a knockout mutation in the abcr gene, see Weng et al., Cell, 98:13-23 (1999), are useful for the study of RmP function as well as for an in vivo screening of the effectiveness for candidate substances. These animals manifest the complex ocular phenotype: (i) slow photoreceptor degeneration, (ii) delayed recovery of rod sensitivity following light exposure, (iii) elevated atRAL and reduced atROL in photoreceptor outer-segments following a photobleach, (iv) constitutively elevated phosphatidylethanolamine (PE) in outer-segments, and (v) accumulation of lipofuscin in RPE cells. See Weng et al., Cell, 98:13-23 (1999).

Rates of photoreceptor degeneration can be monitored in treated and untreated wild-type and abcr−/− mice by two techniques. One is the study of mice at different times by ERG analysis and is adopted from a clinical diagnostic procedure. See Weng et al., Cell, 98:13-23 (1999). An electrode is placed on the corneal surface of an anesthetized mouse and the electrical response to a light flash is recorded from the retina. Amplitude of the a-wave, which results from light-induced hyperpolarization of photoreceptors, is a sensitive indicator of photoreceptor degeneration. See Kedzierski et al., Invest. Opthalmol. Vis. Sci., 38:498-509 (1997). ERGs are done on live animals. The same mouse can therefore be analyzed repeatedly during a time-course study. The definitive technique for quantitating photoreceptor degeneration is histological analysis of retinal sections. The number of photoreceptors remaining in the retina at each time point will be determined by counting the rows of photoreceptor nuclei in the outer nuclear layer.

### Example 8

### Effect of Formula (I) in Combination with a Second Agent on A2E Accumulation

Administration of Formula (I) in combination with a second agent recited herein to an experimental group of mice and administration of DMSO alone to a control group of mice is performed and assayed for accumulation of A2E. The experimental group is given 0.1 mg/kg per day to 1.0 mg/kg per day of isotretinoin with 600 mg vitamin C, 450 mg vitamin E, 30,000 IU vitamin A, 90 mg zinc and 2.5 mg copper in 25 μl of DMSO. Higher dosages of isotretinoin are tested if no effect is seen with the highest dose of 1.0 mg/kg isotretinoin. The control group is given 25 μl injections of DMSO alone. Mice can be implanted with a pump which deliver either experimental or control substances at a rate of 0.25 μl/hr for various experimental time periods not to exceed one month.

To assay for the accumulation of A2E in abcr−/− mice RPE, 0.1 mg/kg per day to 1.0 mg/kg per day of isotretinoin with 600 mg vitamin C, 450 mg vitamin E, 30,000 IU vitamin A, 90 mg zinc and 2.5 mg copper is provided per day via osmotic pump to 3-month old abcr−/− mice. After 1 month, both experimental and control mice are killed and the levels of A2E in the RPE are determined by HPLC.

### Example 9

### Effect of Formula (I) in Combination with a Second Agent on Lipofuscin Accumulation

Administration of Formula (I) in combination with a second agent recited herein to an experimental group of mice and administration of DMSO alone to a control group of mice is performed and assayed for the accumulation of lipofuscin. The experimental group is given 0.1 mg/kg per day to 1.0 mg/kg per day of isotretinoin with 600 mg vitamin C, 450 mg vitamin E, 30,000 IU vitamin A, 90 mg zinc and 2.5 mg copper in 25 μl of DMSO. Higher dosages of isotretinoin are tested if no effect is seen with the highest dose of 1.0 mg/kg isotretinoin. The control group is given 25 μl injections of DMSO alone. Mice can be implanted with a pump which deliver either experimental or control substances at a rate of 0.25 μl/hr for various experimental time periods not to exceed one month.

To assay for the effects of Formula (I) in combination with a second agent recited herein on the formation of lipofuscin in treated and untreated abcr−/− mice, eyes can be examined by electron microscopy.

### Example 10

### Effect of Formula (I) in Combination with a Second Agent on Rod Cell Death or Rod Functional Impairment

Administration of Formula (I) in combination with a second agent recited herein to an experimental group of mice and administration of DMSO alone to a control group of mice is performed the effect on rod cell death or rod functional impairment. The experimental group is given 0.1 mg/kg per day to 1.0 mg/kg per day of isotretinoin with 600 mg vitamin C, 450 mg vitamin E, 30,000 IU vitamin A, 90 mg zinc and 2.5 mg copper in 25 μl of DMSO. Higher dosages of isotretinoin are tested if no effect is seen with the highest dose of 1.0 mg/kg isotretinoin. The control group is given 25 μl injections of DMSO alone. Mice can be implanted with a pump which deliver either experimental or control substances at a rate of 0.25 μl/hr for various experimental time periods not to exceed one month.

Mice treated with isotretinoin and a second agent for approximately 8 weeks can be assayed for the effects of such a treatment on rod cell death or rod functional impairment by monitoring ERG recordings and performing retinal histology.

### Example 11

### Testing for Protection from Light Damage

The following study is adapted from Sieving, P. A., et al, Proc. Natl. Acad. Sci., 98:1835-40 (2001). For chronic light-exposure studies, Sprague-Dawley male 7-wk-old albino rats are housed in 12:12 h light/dark cycle of 5 lux fluorescent white light. Injections of 0.1 mg/kg per day to 1.0 mg/kg per day of isotretinoin with 600 mg vitamin C, 450 mg vitamin E, 30,000 IU vitamin A, 90 mg zinc and 2.5 mg copper i.p. in 0.18 ml DMSO are given three times daily to chronic rats for 8 wk. Controls receive 0.18 ml DMSO i.p. Rats are killed 2 d after final injections. Higher dosages of isotretinoin are tested if no effect is seen with the highest dose of 1.0 mg/kg isotretinoin.

For acute light-exposure studies, rats are dark-adapted overnight and given a single i.p. injection of 0.1 mg/kg per day to 1.0 mg/kg per day of isotretinoin with 600 mg vitamin C, 450 mg vitamin E, 30,000 IU vitamin A, 90 mg zinc and 2.5 mg copper in 0.18 ml DMSO under dim red light and kept in darkness for 1 h before being exposed to the bleaching light before ERG measurements. Rats exposed to 2,000 lux white fluorescent light for 48 h. ERGs are recorded 7 d later, and histology is performed immediately.

Rats are euthanized and eyes are removed. Column cell counts of outer nuclear layer thickness and rod outer segment (ROS) length are measured every 200 μm across both hemispheres, and the numbers are averaged to obtain a measure of cellular changes across the entire retina. ERGs are recorded from chronic rats at 4 and 8 wks of treatment. In acute rodents, rod recovery from bleaching light is tracked by dark-adapted ERGs by using stimuli that elicit no cone contribution. Cone recovery is tracked with photopic ERGs. Prior to ERGs, animals are prepared in dim red light and anaesthetized. Pupils are dilated and ERGs are recorded from both eyes simultaneously by using gold-wire corneal loops.

### Example 12

### Combination Therapy Involving Compounds of Formula (I) and a Second Agent with a Nitric Oxide Inducer

Mice and/or rats are tested in the manner described in Examples 8-11, but with an additional two arms. In one of the additional arms, groups of mice and/or rats are treated with a suitable nitric oxide inducer which can include currently available statins such as: Lipitor® (Atorvastatin), Mevacor® (Lovastatin), Pravachol® (Pravastatin sodium), Zocor™ (Simvastatin), Leschol (fluvastatin sodium) and the like with optimal dosage based on weight. In the second additional arm, groups of mice and/or rats are treated with a combination of 1.0 mg/kg per day of isotretinoin with 600 mg vitamin C, 450 mg vitamin E, 30,000 IU vitamin A, 90 mg zinc and 2.5 mg copper and increasing doses of the statin used in the previous step. Suggested human dosage of such statins are for example: Lipitor® (Atorvastatin) 10-80 mg/day, Mevacor® (Lovastatin) 10-80 mg/day, Pravachol® (Pravastatin sodium) 10-40 mg/day, Zocor™ (Simvastatin) 5-80 mg/day, Leschol (fluvastatin sodium) 20-80 mg/day. Dosage of statins for mice and/or rat subjects should be calculated based on weight. The benefits of the combination therapy are assayed as described in Examples 8-11

### Example 13

### Timing of Administration of the Components of Formula (I) and a Second Agent

Mice and/or rats are tested in the manner described in Examples 8-11, but with an additional three arms. In one of the additional arms, groups of mice and/or rats are treated with the a second agent recited herein prior to the administration of Formula (I). In the second additional arm, groups of mice and/or rats are treated with a second agent recited herein during the administration of Formula (I). In the third additional arm, groups of mice and/or rats are treated with a second agent recited herein after the administration of Formula (I). The supplemental concentrations can vary from 50 mg to about 600 mg vitamin C, 20 IU to about 450 mg vitamin E, 900 IU to about 30,000 IU vitamin A, 10 mg to about 90 mg zinc, and 0.5 mg to about 2.5 mg copper. The Formula (I) concentration can range from 0.1 mg/kg/day to 1.0 mg/kg/day. The benefits of the timing of administration are assayed as described in Examples 8-11.

All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-119. (canceled)

120. A method for reducing the formation of drusen in an eye of a human comprising administering to the mammal at least once:
a. an effective amount of a first agent, wherein the first agent has the structure 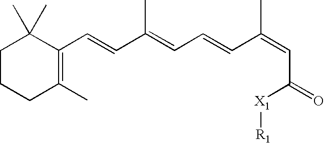
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoralkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof; and
b. an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their derivatives.

121. A method for treating macular degeneration in an eye of a human comprising administering to the mammal an effective amount of a compound comprising:
a. an effective amount of a first agent, wherein the first agent has the structure 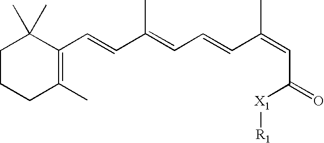
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoralkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof; and
b. an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their derivatives.

122. The method of claim 121 wherein the macular degeneration is dry form age-related macular degeneration.

123. The method of any of claims 120-121, wherein the effective amount of the first agent is systemically administered to the mammal.

124. The method of any of claims 120-121, wherein the second agent comprises an antioxidant.

125. The method of claim 124, wherein the antioxidant is selected from the group consisting of coenzyme Q, 4-hydroxy-2,2,6,6-tetramethylpiperidine-N-oxyl, lutein, butylated hydroxytoluene, resveratrol, a trolox analogue, and bilberry extract.

126. The method of any of claims 120-121, wherein the second agent comprises a mineral.

127. The method of claim 126, wherein the mineral is selected from the group consisting of a copper-containing mineral, a zinc-containing mineral, and a selenium-containing compound.

128. The method of any of claims 120-121, wherein the second agent comprises an inducer of nitric oxide production.

129. The method of claim 128, wherein the inducer of nitric oxide production is a statin.

130. The method of any of claims 120-121, wherein the second agent is an additional anti-inflammatory agent.

131. The method of any of claims 120-121, wherein the additional agent is a negatively charged phospholipid.

132. The method of claim 131 wherein the negatively charged phospholipid is selected from the group consisting of phosphatidylglycerol, lutein and zeaxanthin.

133. The method of any of claim 120-121, wherein the additional agent is a derivative of an isomer of 13-cis-retinoic acid.

134. A method for treating atrophy of the pigmented epithelium of the retina and photoreceptors in an eye of a human comprising administering to the mammal at least once:
a. an effective amount of a first agent, wherein the first agent has the structure 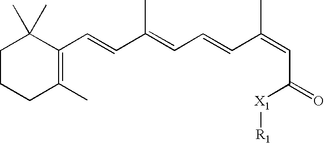
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoralkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof; and
b. an effective amount of a second agent comprising an agent selected from the group consisting of an antioxidant, a mineral, an inducer of nitric oxide production, an anti-inflammatory agent, a negatively charged phospholipid, and isomers of 13-cis-retinoic acid and their derivatives.
